(19) United States
(12) Patent Application Publication
(10) Pub. No.: US 2013/0131164 A1
(43) Pub. Date: May 23, 2013

(54) STABILIZED PHENYLCARBAMATE DERIVATIVE IN SOLID STATE

(71) Applicants: Ernesto Duran Lopez, Castellbisbal (ES); Jordi Bosch I Lladó, Girona (ES); Judit Serra Miralles, Girona (ES)

(72) Inventors: Ernesto Duran Lopez, Castellbisbal (ES); Jordi Bosch I Lladó, Girona (ES); Judit Serra Miralles, Girona (ES)

(73) Assignee: MEDICHEM, S.A., Sant Joan Despi (ES)

(21) Appl. No.: 13/579,639

(22) PCT Filed: Feb. 18, 2011
(86) PCT No.: PCT/EP2011/052469

§ 371 Date: Sep. 24, 2012

Related U.S. Application Data
(60) Provisional application No. 61/306,165, filed on Feb. 19, 2010.
(60) Provisional application No. 61/306,196, filed on Feb. 19, 2010.

(51) Int. Cl. C07C 271/28

(52) U.S. Cl. CPC C07C 271/28
USPC 514/485; 560/27

(57) ABSTRACT

The invention relates to retigabine with improved color quality, and to processes for preparing the same. In addition, the invention relates to a process for drying wet retigabine. Also, the invention relates to stabilized or substantially stabilized retigabine in solid state, or a mixture or pharmaceutical formulation comprising the same. Further, the invention also relates to an improved process for preparing retigabine.

This application claims the benefit of the U.S. Provisional Patent Application Ser. No. 61/306,196 filed on 19 Feb. 2010 and U.S. Provisional Patent Application Ser. No. 61/306,165 filed on 19 Feb. 2010.

### FIELD OF THE INVENTION

The invention relates to retigabine with improved color quality, and to a process for preparing the same. In addition, the invention relates to a process for drying wet retigabine. Also, the invention relates to stabilized or substantially stabilized retigabine in solid state, or a mixture or pharmaceutical formulation comprising the same. Further, the invention also relates to an improved process for preparing retigabine.

### BACKGROUND ART

Retigabine, which is a compound of formula (I) below, is the international commonly accepted non-proprietary name for ethyl 2-amino-4-(4-fluorobenzylamino)phenylcarbamate.

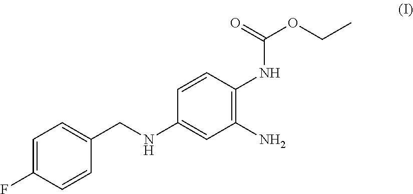 [SMILES: CCOC(=O)Nc1ccc(NCc2ccc(F)cc2)cc1N]

Retigabine has an empirical formula of C₁₆H₁₈FN₃O₂, and a molecular weight of 303.33 g/mol. Retigabine is a pharmaceutical substance with anticonvulsive, antipyretic and analgesic activity, and can thus be employed in pharmaceutical preparations. In the United States of America, the non-proprietary name retigabine has been superseded by ezogabine.

U.S. Pat. No. 5,384,330 discloses the preparation of retigabine and similar compounds. More precisely, Example 1 of U.S. Pat. No. 5,384,330 describes the preparation of retigabine, which is isolated in dihydrochloride form.

CNS Drug Rev. 2005, 11, 1 describes the dihydrochloride salt of retigabine to be hygroscopic and unstable under medium to long-term storage at −18° C. It also describes that retigabine is preferably stored as the free base, isolated from light. The tendency of retigabine to easily oxidize upon contact with air during solubilization as the free base is also described.

U.S. Pat. No. 6,538,151 discloses the isolation of retigabine in form of the free base. Retigabine is obtained by crystallization from ethanol, n-butanol, toluene or isopropanol. Retigabine is also obtained by digestion in toluene, acetone, or ethanol. Wet retigabine is dried to weight constancy at about 50° C. to 55° C. under vacuum. No details are provided regarding the color of the retigabine obtained. Further, U.S. Pat. No. 6,538,151 also describes three different crystalline forms of retigabine, denominated therein as modifications A, B, and C, and also provides processes for preparing the same.

Retigabine is described in Indian J. Pharmacol. 2005, 37, 340, as a purple colored compound.

Furthermore, the inventors have also now observed that retigabine with low color quality (i.e., low “whiteness” quality) is obtained by reproduction of the processes already described in the prior art. There is a need, therefore, to improve the color quality of retigabine that is obtained by prior art processes, which color quality is considered as indicative of the presence of specific impurities that may or may not be detectable by conventional methods such as HPLC. Substantially as hereinbefore described, retigabine is an active pharmaceutical substance that can be employed in pharmaceutical preparations and as such it is necessary to obtain retigabine as a high purity product with a minimum amount of undesired by-products. In particular, the presence of impurities may adversely affect the safety and shelf life of formulations comprising retigabine.

U.S. Pat. No. 5,384,330 discloses the preparation of retigabine and similar compounds. More precisely, Example 1 of U.S. Pat. No. 5,384,330 describes two alternatives for the synthesis of retigabine. In variant A of Example 1 of U.S. Pat. No. 5,384,330, 2-amino-5-(4-fluorobenzylamino)nitrobenzene or 2-amino-4-(4-fluorobenzylamino)nitrobenzene is hydrogenated in the presence of Raney nickel (as illustrated in Scheme 1 below). Once the hydrogenation is completed, the catalyst is filtered off and the filtrate is reacted with ethyl chloroformate in the presence of diisopropylethylamine to obtain retigabine.

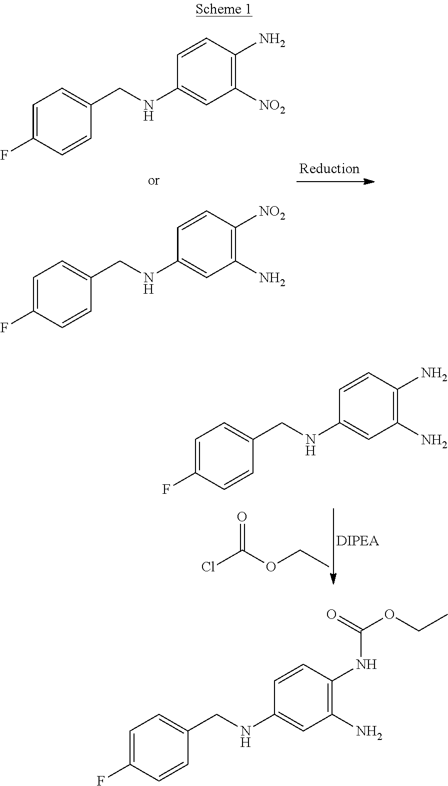

In variant B of Example 1 of U.S. Pat. No. 5,384,330, 2-ethoxycarbonylamino-5-(4-fluorobenzylamino)-nitrobenzene is hydrogenated in the presence of Raney nickel to obtain retigabine (as illustrated in Scheme 2 below).

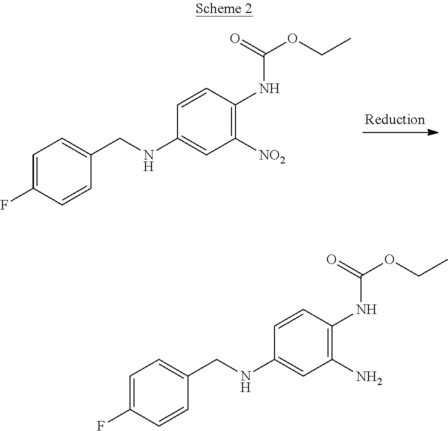 [SMILES: CCOC(=O)Nc1ccc(NCc2ccc(F)cc2)cc1[N+](=O)[O-] ; CCOC(=O)Nc1ccc(NCc2ccc(F)cc2)cc1N]

U.S. Pat. No. 5,384,330 further describes that catalytic hydrogenation is particularly suitable for the reduction step of the above processes using Raney nickel as catalyst, as well as other precious metals such as palladium and platinum. Reduction with Zn/HCl, Sn/HCl and Fe/HCl is also described to be a suitable process for the reduction reaction. Other suitable reagents described in U.S. Pat. No. 5,384,330 for the reduction step are salts of H₂S in alcohol/water, activated aluminum in aqueous ether, SnCl₂/HCl, and ammonium formate.

Activated metal catalysts, such as Raney nickel, that are described in the prior art for reduction of retigabine as explained above, are pyrophoric materials that have to be handled under an inert atmosphere. Furthermore, these catalysts also contain significant amounts of adsorbed hydrogen gas after the reduction process and this further requires handling under an inert atmosphere to avoid ignition when exposed to air.

A still further consideration with the prior art processes is that hydrogen gas is an extremely flammable reagent which explodes upon ignition in contact with oxygen, and again the presence of an inert atmosphere is thus required. Since air mixtures containing only about 4% of hydrogen are flammable, special reactors and isolation systems are needed for carrying out reactions under hydrogen atmosphere at industrial scale. Furthermore, hydrogen gas is commonly stored in compressed gas cylinders, which are also dangerous due to the risk of explosive expansion of the compressed gas under mechanical stress. Therefore, use of hydrogen gas is not recommended at industrial scale.

When carrying out the processes of the prior art it is also necessary to take into consideration that the metals that have been used as process catalysts or reagents during the synthesis of pharmaceutical compounds, such as retigabine, may result in metal residues in the final pharmaceutical substance, and consequently in the final drug product. Such metal residues do not provide any therapeutic benefit to the patient and should, therefore, be evaluated and restricted in view of safety- and quality-based criteria. Maximum acceptable concentration limits of metal residues in the pharmaceutical substances are regulated by the European Medicines Agency in the “Guideline on the Specification Limits for Residues of Metal Catalysts or Metal Reagents”, reference EMEA/CHMP/SWP/4446/2000. Nickel, palladium and platinum are classified as metals of significant safety concern and furthermore are considered as known or suspected human carcinogens, or possible causative agents of other significant toxicity.

The inventors have now found that retigabine with a very high content of residual nickel is obtained by following the synthetic processes described in the prior art. Even after usual purification procedures such as recrystallization, levels of residual nickel in retigabine are much higher than the concentration limits accepted by the regulatory guidelines, and thus further purification steps and/or the use of metal scavengers are needed, which is particularly detrimental for both environmental and economic points of view. Using precious metal catalysts (e.g., palladium on charcoal) as alternative catalysts for the hydrogenation process, the inventors have also found levels of residual metals in retigabine, which are higher than the concentration limits accepted by the regulatory guidelines.

Furthermore, in both examples of U.S. Pat. No. 5,384,330, retigabine is isolated in the form of its dihydrochloride salt. CNS Drug Rev. 1996, 2, 308 describes that the synthetic purity of the free base form of retigabine is greater than that of the corresponding dihydrochloride. On the other hand, CNS Drug Rev. 2005, 11, 1 describes the dihydrochloride salt of retigabine to be hygroscopic and unstable under medium to long-term storage at −18° C., due to the formation of significant amounts of the ring-closed product 5-(4-fluorobenzylamino)-1,3-dihydrobenzimidazol-2-one and small amounts of uncharacterized oxidized products. Therefore, for chemical and technological reasons, retigabine free base was chosen for further development of the pharmaceutical form.

In view of the above, there is a need for improved processes of preparing retigabine, in particular retigabine free base, and also for improved processes of preparing key intermediate compounds useful in preparing retigabine. Additionally, the preparation of retigabine substantially free of metal residues associated with prior art preparation techniques would be advantageous. Such improved processes and retigabine prepared thereby are now provided by the present invention substantially as hereinafter described.

### BRIEF SUMMARY OF THE INVENTION

The present invention relates to phenylcarbamate derivatives, such as retigabine, having improved color quality. Additionally, the present invention provides processes of preparing phenylcarbamate derivatives, such as retigabine, having improved color quality.

The invention provides retigabine with improved color quality. For example, in an embodiment the invention provides retigabine as a white or substantially white color. The invention also provides retigabine having a Whiteness Index in the range of about 60 to 100. The invention further provides retigabine characterized by an L* color coordinate in the range of about 90 to 100. The invention further provides retigabine characterized by an a* color coordinate in the range of about −1.00 to +1.00. The invention further provides retigabine characterized by a b* color coordinate in the range of about −7.00 to +7.00.

In other embodiments, the present invention provides a process of determining the quantitative color and/or purity of retigabine.

The invention also provides a process of improving the color quality of retigabine.

The invention further provides a process for drying wet retigabine.

In another embodiment the invention provides stabilized or substantially stabilized retigabine in solid state or pharmaceutical formulation comprising said stabilized retigabine in solid state.

In another embodiment, the present invention provides a process of synthesizing stabilized or substantially stabilized retigabine in solid state.

In yet another embodiment, the present invention provides a process for formulating stabilized or substantially stabilized retigabine in solid state.

In a further embodiment, the invention provides a process of storing or packaging retigabine in solid state or pharmaceutical formulation comprising said retigabine in solid state.

The invention also provides a process of stabilizing retigabine in solid state or pharmaceutical formulation comprising said retigabine in solid state.

The invention further provides a process of controlling the color quality or Whiteness Index and/or the HPLC chemical purity of retigabine.

The present invention also relates to an improved process for preparing phenylcarbamate derivatives, such as retigabine, and in particular retigabine free base. Additionally, the present invention provides retigabine, preferably retigabine free base, substantially free of metal residues of significant or low safety concern, and in particular retigabine, preferably retigabine free base, substantially free from any of nickel, palladium or platinum.

### DETAILED DESCRIPTION OF THE INVENTION

The inventors have now surprisingly found that retigabine, especially the free base thereof, can be obtained according to the present invention as a white or substantially white solid. According to the present invention, therefore, there is provided retigabine with white color.

The present invention further involves assessing the purity of retigabine by means of a quantitative colorimetric measurement of solid retigabine. The method includes using a colorimeter or spectrophotometer apparatus to measure the L*, a* and b* coordinates of a solid sample of retigabine. Thus, the color of the solid sample is located in the CIE 1976 L*, a*, b* Color Space (CIELAB; CIE stands for Commission Internationale de l'Éclairage or International Commission on Illumination). The three parameters in the model represent the lightness of the color (i.e., L*, an L*=0 indicates black and L*=100 indicates white), its position between magenta and green (i.e., a*, negative values indicate green while positive values indicate magenta) and its position between yellow and blue (i.e., b*, negative values indicate blue and positive values indicate yellow).

The color quality (i.e., “whiteness”) of retigabine is corroborated by the colorimetric measurements that yield values in the CIELAB color space that are very closed to the values of absolute white, namely L*=100, a*=0, b*=0. This can be further understood by reference to the US Pharmacopoeia 32^{nd} ed., General Chapter 1061, p. 512.

The Whiteness Index (WI) of retigabine is calculated according to ASTM E313-05 “Standard Practice for Calculating Yellowness and Whiteness Indices from Instrumentally Measured Color Coordinates” using the following formula:

WI=Y+(WI,x)(xₙ−x)+(WI,y)(yₙ−y)

where: xₙ and Yₙ are the chromaticity coordinates for the CIE Standard illuminant and source used, WI,x and WI,y are numerical coefficients, and Y, x, and y are the luminance factor and the chromaticity coordinates of the specimen (which can be derived from the L*, a*, and b* coordinates for a given illuminant and measurement geometry). Values for all these variables (except those measured for the specimen), for illuminant D65 (daylight) and a 2° angle of observation, are provided in Table 1.

TABLE 1

| | |
|---|---|
| xₙ | 0.3127 |
| yₙ | 0.3290 |
| WI, x | 800 |
| WI, y | 1700 |

A perfect white is a Perfect Reflecting Diffuser (PRD), which has a Whiteness Index value of 100. Pressed magnesium oxide (MgO) and pressed barium sulfate (BaSO₄) are high-reflectance materials that closely approximate a PRD.

Retigabine according to the present invention preferably has a Whiteness Index in the range of about 60 to 100, measured using illuminant D65 (daylight) and a 2° angle of observation, and more preferably retigabine according to the present invention has a Whiteness Index in the range of about 70 to 100, measured using illuminant D65 (daylight) and a 2° angle of observation. It is preferred that retigabine according to the present invention can be further characterized by an L* color coordinate in the range of about 90 to 100, preferably about 94 to 100. Furthermore, it is preferred that retigabine according to the present invention can be characterized by an L* color coordinate in the range of about 90 to 100, an a* color coordinate in the range of about −1.00 to +1.00 and b* color coordinate in the range of about −7.00 to +7.00, measured using illuminant D65 (daylight) and a 2° angle of observation. Still more preferably, retigabine according to the present invention can be characterized by an L* color coordinate in the range of about 94 to 100, an a* color coordinate in the range of about −0.70 to +0.70 and b* color coordinate in the range of about −6.00 to +6.00, measured using illuminant D65 (daylight) and a 2° angle of observation.

Advantageously, retigabine according to the present invention has a purity greater than approximately 99.5% as measured by HPLC.

Preferably, retigabine according to the present invention is provided in the form of retigabine free base.

According to the present invention, there is further provided a process of determining the quantitative color and/or purity of retigabine, which process comprises measuring the white index and/or color coordinates L*, a* and b* of a retigabine sample in accordance with the CIE 1976 L*, a*, b* Color Space. Preferably in the case of a purity measurement this involves correlating the white index and/or color coordinates L*, a* and b* obtained with a reference sample.

There is also provided by the present invention a process of improving the color quality of retigabine, preferably retigabine-free base, which process comprises crystallizing or slurrying retigabine, preferably retigabine-free base, with a solvent mixture comprising at least one alcoholic solvent and at least one non-alcoholic solvent, and isolating retigabine, preferably retigabine-free base, from a resulting mixture.

The alcoholic solvent can be methanol, ethanol, n-propanol, isopropanol or n-butanol, preferably isopropanol. The non-alcoholic solvent can be an aromatic solvent, an alkyl-type solvent, an ester, a ketone, an ether or a nitrile, preferably an ester-type solvent, and more preferably ethyl acetate.

It is particularly preferred to crystallize or slurry retigabine, preferably retigabine free base, with a mixture of isopropanol and ethyl acetate in order to provide retigabine with enhanced color quality (i.e., “whiteness”), compared with retigabine as prepared by processes already described in the prior art (See Test 1, Color Measurement, below).

The inventors have also found that the color quality (i.e., “whiteness”) of retigabine can be worsened under particular drying conditions, which color quality is considered as indicative of the presence of specific impurities that may or may not be detectable by conventional methods such as HPLC. More specifically, the inventors have observed worsening of color quality when wet retigabine is heated in the prior art processes to a temperature between 50° C. to 55° C., to promote solvent removal and/or to accelerate the solvent removal process under reduced pressure. The inventors have further observed degradation of wet retigabine at temperatures described for the drying conditions in the prior art processes (i.e., 50° C. to 55° C.), even under inert atmosphere and/or reduced pressure. Degradation has been observed by the inventors by monitoring substantial color worsening under these conditions (See Tests 2-4, Color Measurements, below). Further, surprisingly said degradation has not been observed by conventional HPLC method (See Tests 2-4, HPLC purity Measurements, below), since the chemical purity, as measured by conventional HPLC method, indicates no apparent chemical worsening of the retigabine, but a deceitful constant HPLC purity value. Thus, degradation at temperatures described for the drying conditions in the prior art processes (i.e., 50° C. to 55° C.) is observed in connection with one or more unknown specific impurities that are not detectable by conventional HPLC methods.

The present invention thus further provides an improved process for drying wet retigabine, preferably retigabine-free base, with a reduced deleterious effect on the color quality (i.e., “whiteness”) of the product, while keeping the HPLC chemical purity stability, which comprises drying wet retigabine at a temperature below about 50° C., preferably below about 48° C., more preferably below about 45° C., more preferably below about 43° C., more preferably below about 40° C., more preferably below about 38° C., and even more preferably below about 35° C. The wet retigabine is preferably provided by dissolving or suspending retigabine, preferably retigabine-free base, in a suitable solvent, and isolating the wet retigabine.

Accordingly the present invention further provides a process for improving the color quality of retigabine, preferably retigabine-free base, by dissolving or suspending retigabine, preferably retigabine-free base, in a suitable solvent, isolating wet retigabine, and drying the wet retigabine, preferably retigabine free base, at a temperature below about 50° C., preferably below about 48° C., more preferably below 45° C., more preferably below about 43° C., more preferably below about 40° C., more preferably below about 38° C., and even more preferably below about 35° C.

Preferably, the processes above comprise drying wet retigabine at a temperature in the range of about 10 to 50° C., preferably in the range of about 10 to 45° C., more preferably in the range of about 15 to 40° C., and more preferably in the range of about 20 to 35° C., and further preferably under reduced pressure. The reduced pressure is preferably lower than 1013 hPa, preferably lower than 700 hPa, more preferably lower than 500 hPa, even more preferably lower than 250 hPa, and even more preferably lower than 100 hPa. Preferably, the dissolving or suspending retigabine comprises crystallizing or slurrying retigabine, preferably retigabine-free base, according to the process of the invention above.

The term “wet retigabine”, as described herein is meant to describe retigabine having certain percentage of one or more residual solvents. The total percentage of one or more residual solvents in the wet retigabine can be between about 70% (w/w) and about 0.1% (w/w), with respect to the total weight of the wet retigabine. For example the total percentage of residual solvent in the wet retigabine can be equal to or less than 70% (w/w), equal to or less than 50% (w/w), equal to or less than 25% (w/w), equal to or less than 10% (w/w), equal to or less than 5% (w/w), equal to or less than 1% (w/w), or equal to or less than 0.5% (w/w), and equal to or more than 0.1% (w/w), with respect to the total weight of the wet retigabine. Accordingly, the term “dry retigabine”, as described herein is meant to describe retigabine having less than 0.1% (w/w) of total residual solvents.

The suitable solvent of the process described hereinabove and any residual solvent described hereinabove can be any solvent known in the art which may be inert and suitable for dissolving or suspending retigabine, especially retigabine free base. For example, the solvent can be acetone, acetonitrile, allyl alcohol, pentyl acetate, tert-pentyl alcohol, anisole, benzene, benzyl alcohol, bromobenzene, 1,3-butanediol, 1,4-butanediol, 1-butanol, 2-butanol, tert-butanol, 2-butoxyethanol, butyl acetate, butyl ether, chlorobenzene, chloroform, cyclohexane, cyclohexanol, cyclohexanone, cyclopentane, cyclopentyl methyl ether, 1,2-dichlorobenzene, 1,2-dichloroethane, dichloromethane, diethoxymethane, diethylene glycol, diethylene glycol diethyl ether, diethylene glycol ethyl ether, diethylene glycol ethyl ether acetate, diethylene glycol hexyl ether, diethylene glycol methyl ether, diisobutyl ketone, 1,2-dimethoxyethane, dimethoxymethane, N,N-dimethylacetamide, N,N-dimethylformamide, dimethylsulfoxide, 1,4-dioxane, dipropylene glycol, dipropylene glycol methyl ether, ethanol, 2-ethoxyethanol, ethyl acetate, ethylbenzene, ethylene glycol, ethylene glycol diethyl ether, ethyl ether, formamide, glycerol, n-heptane, n-hexane, 1-hexanol, hexylene glycol, isoamyl alcohol, isobutanol, isobutyl acetate, isopropanol, isopropyl acetate, isopropyl ether, methanol, 2-methoxyethanol, 2-methoxyethyl acetate, 2-methoxyethyl ether, methyl acetate, methyl tert-butyl ether, methyl cyclohexane, methyl ethyl ketone, methyl formate, methyl isobutyl ketone, N-methylpyrrolidone, 2-methyltetrahydrofuran, nitrobenzene, nitromethane, 1-octanol, n-pentane, 1-pentanol, 3-pentanone, 1-propanol, propyl acetate, propylene glycol, propylene glycol methyl ether, propylene glycol methyl ether acetate, propylene oxide, pyridine, 2-pyrrolidone, tetrachloroethylene, tetraethylene glycol, tetrahydrofuran, toluene, 1,1,1-trichloroethane, trichloroethylene, triethylene glycol, triethylene glycol dimethyl ether, water, xylene, or mixtures thereof. Preferably, the solvent is a C₁-C₈ alcohol solvent, a C₃-C₈ ketone solvent, a C₃-C₁₀ ester solvent, an aromatic hydrocarbon solvent, or any mixture thereof. More preferably, the solvent is ethanol, isopropanol, n-butanol, acetone, ethyl acetate, toluene, or mixtures thereof.

It is also known from the prior art that retigabine is light sensitive in solid form, since it is described that retigabine is preferably isolated from light on storage. The inventors have now also found that retigabine in solid state, especially retigabine-free base, is susceptible to oxidation upon contact with the atmosphere, which has not previously been disclosed in the prior art.

Although the prior art already describes the tendency of retigabine free base to easily oxidize upon contact with air when dispersed into the unstructured environment of a solution, this is not an indication that the retigabine when present in solid state, which hence presents an ordered crystalline habit, may also show susceptibility to oxidation. That is, it is known that the susceptibility to oxidation of a compound in solid form has nothing to do with its susceptibility when dispersed within a solution. In this regard, it is understood from the prior art that an oxidizing agent needs to be present in a system to promote oxidation in solid form. Molecular oxygen from the atmosphere has been shown to react with some organic crystals, but this reactivity depends on the crystal form (morphology), which governs the permeability and solubility of oxygen in the crystal matrix. In most cases, however, reactions of crystalline materials with molecular oxygen do not produce oxidation products that can fit into the crystalline lattice, whereby the reaction is hindered energetically and oxidation in solid state is not observed (see Pharm. Dev. Technol. 2002, 7, 1-32).

The inventors have now surprisingly found that the stability of retigabine, especially retigabine-free base, in solid state is dramatically affected by the oxygen content in a surrounding environment. Degradation is particularly observed by both worsening of the color quality (i.e., “whiteness”) [i.e. at least about 55% of WI worsening after 7 days exposure to air atmosphere at room temperature] and worsening of the chemical HPLC purity (i.e. at least about 1% of HPLC chemical purity worsening after 7 days exposure to air atmosphere at room temperature) of solid retigabine in contact with atmospheric oxygen (See Test 5, Color and HPLC Purity Measurements, below). Specifically, the inventors have observed that, after 7 days exposure to air atmosphere at room temperature, HPLC purity of solid retigabine is decreased from 99.9% to 99.0%, rendering said solid retigabine not suitable for pharmaceutical use (i.e. generally, the standard of purity for Active Pharmaceutical Ingredients is not less than 99.5%). In particular, the inventors have found that the deleterious effect of air on the color quality and on the HPLC chemical purity of retigabine is dramatically higher than the effect of light on the color quality and the chemical HPLC purity of retigabine. Surprisingly, when keeping the same samples of solid retigabine under an inert atmosphere (i.e. nitrogen atmosphere) even in the presence of light, no substantial worsening of the color quality was observed (i.e. only 1% WI worsening after 7 days exposure to inert atmosphere at room temperature) and in addition no substantial HPLC purity worsening was detected (See Test 5, Color and HPLC Purity Measurements, below).

The present invention, therefore, further provides an improvement in stability of retigabine in solid state according to the present invention as illustrated by an improvement in both the color quality and the HPLC chemical purity quality thereof substantially as herein described.

Accordingly, the present invention provides retigabine in solid state, preferably retigabine-free base, exposed to an inert gas atmosphere. The present invention further provides retigabine in solid state, preferably retigabine-free base, stabilized or substantially stabilized by exposure to an inert gas atmosphere. Suitably, the retigabine in solid state, preferably retigabine-free base, is stabilized or substantially stabilized by storage in an enclosed container or packaging wherein inert gas atmosphere is provided within the container or packaging.

The terms “retigabine” and “retigabine in solid form or state”, as described herein, are meant to refer to dry solid retigabine in any known crystalline form, mixtures thereof, or mixtures thereof with amorphous form. Also, the term “wet retigabine”, as described herein, is meant to refer to wet solid retigabine in any known crystalline form, mixtures thereof, or mixtures thereof with amorphous form. Specifically, by wet or dry retigabine in any known crystalline form, mixtures thereof, or mixtures thereof with amorphous form is understood that the total content of retigabine, especially retigabine-free base, contains at least 1% of a known crystalline form, or of mixtures thereof. That is, the wet or dry retigabine, especially retigabine-free base, in solid state as described in the invention can contain up to 99% of amorphous form, with reference to the total retigabine (dry content), especially retigabine-free base. Preferably, the wet or dry retigabine, especially retigabine-free base, in solid state can contain up to 90% of amorphous form, more preferably can contain up to 80% of amorphous form, more preferably can contain up to 70% of amorphous form, more preferably can contain up to 60% of amorphous form, more preferably can contain up to 50% of amorphous form, more preferably can contain up to 10% of amorphous form, more preferably can contain up to 1% of amorphous form, and even more preferably can contain up to 0.1% of amorphous form, with reference to the total retigabine (dry content), especially retigabine-free base. Namely, the retigabine, especially retigabine-free base, can contain ranges between 0.1 to 99%, of amorphous form with reference to the total retigabine (dry content), especially retigabine-free base. Preferably, the wet or dry retigabine, especially retigabine-free base is in crystalline form, meaning that it does not contain amorphous form or that at least it does not contain detectable amounts of amorphous form. The retigabine-free-base in crystalline form as described herein can be any of the retigabine-free-base known crystalline forms or mixtures thereof (i.e. retigabine known forms A, B, C, and mixtures thereof). In this regard, degradation of the color quality (i.e., “whiteness”) and of the chemical HPLC purity of retigabine in solid state in contact with atmospheric oxygen as described herein is not observed with reference to the crystalline characteristics of the said retigabine. Namely, retigabine, especially retigabine-free base, in solid state, as described herein, does not show relevant changes in its crystalline content and composition when put in contact with atmospheric oxygen as herein described.

There is also provided by the present invention a process of synthesizing retigabine in solid state, especially retigabine-free base, wherein at least formation of retigabine, especially retigabine free base, is carried out under an inert gas atmosphere.

Accordingly, the present invention further provides a pharmaceutical preparation comprising a pharmaceutical formulation comprising retigabine, especially retigabine-free base, in solid state wherein said retigabine is exposed to an inert gas atmosphere. Suitably, the formulation is preferably in an enclosed container or packaging and the inert gas may be provided in the container or packaging.

There is also preferably provided a process of formulating retigabine in solid state, especially retigabine-free base, wherein formulation is carried out in an inert gas atmosphere.

There is also preferably provided a process for providing (i.e. storing or packaging) retigabine in solid state, especially retigabine-free-base or a pharmaceutical formulation comprising retigabine in solid state, in an enclosed container or packaging, wherein a packaging or enclosure procedure is carried out in an inert gas atmosphere.

Furthermore, there is provided a process of stabilizing retigabine in solid state, especially retigabine-free base or a formulation containing the same, which process comprises storing said retigabine or formulation containing the same in an inert gas atmosphere. Suitably, the storing is carried out in an enclosed container or packaging wherein inert gas atmosphere is provided within the container or packaging.

Finally, there is provided a process of controlling both the color quality and/or Whiteness Index and the HPLC chemical purity quality of retigabine in solid state, especially retigabine free base or a pharmaceutical formulation comprising retigabine in solid state, which process comprises storing said retigabine, or formulation containing the same in an inert gas atmosphere. Suitably, the storing is carried out in an enclosed container or packaging wherein inert gas atmosphere is provided within the container or packaging. The term “process of controlling the color quality and/or the HPLC chemical purity quality” is meant to refer that the process is able to control that the white color value and/or the HPLC chemical purity value of the retigabine in solid state do not substantially change for at least one day, preferably at least 5 days, preferably at least 7 days, after the storage described herein. It is understood that the WI and HPLC values do not substantially change when they do not vary more than 0.5%, preferably not more than 0.3%, and preferably not more than 0.1%, with reference the initial value.

The terms “inert gas atmosphere” or “inert atmosphere” as used herein are understood to describe an atmosphere which is essentially free of oxygen, so that the atmosphere is inert with the retigabine in solid state. By essentially free of oxygen is meant to refer that the oxygen content within the surrounding environment (e.g. headspace in the closed container or packaging) is less than 10%, preferably less than 5%, preferably less than 3%, preferably less than 1%, preferably less than 0.1%, more preferably less than 0.01%, and even more preferably is free of oxygen, as measured by partial pressure of oxygen with respect to total pressure of the environment.

The inert atmosphere (e.g. within the enclosed container or packaging) can be generated by adding a suitable amount of an oxygen scavenger (i.e. an agent that reacts with oxygen) under air or synthetic air atmosphere so that to ensure that the atmosphere becomes essentially free of oxygen and hence is inert. Alternatively, the inert atmosphere can be generated by providing vacuum or an inert gas, and optionally further adding a suitable amount of an oxygen scavenger, so that to ensure that the atmosphere is essentially free of oxygen. Also, the inert atmosphere can be generated by admixing directly the retigabine with a suitable amount of an inert antioxidant under air or synthetic air atmosphere, vacuum, or inert gas, so that to ensure that the inert antioxidant reacts with oxygen present in the atmosphere and hence the atmosphere becomes essentially free of oxygen.

The inert gas atmosphere provided in the retigabine, especially retigabine-free base or pharmaceutical formulation thereof, in solid state in an enclosed container or packaging, as hereinbefore described, is preferably provided by the presence of a suitable amount of an oxygen scavenger; and/or by the provision of vacuum or of an inert gas, in the said enclosed container or packaging.

The inert gas substantially as hereinbefore described is preferably nitrogen, a noble gas such as argon, or carbon dioxide.

The oxygen scavenger as herein described is preferably a chemical compound that reacts with oxygen, and more preferably is an iron powder compound or ascorbic acid. Even more preferably, the iron powder compound is an Ageless™ sachet.

In order to avoid oxidation of retigabine in solid state, it should be contended not only with the oxygen available in the headspace of the container or packaging as herein described, but also with the oxygen permeation through the container or packaging walls and caps.

Therefore, it is preferred that the enclosed container or packaging described herein may be a substantially gas exchange non-permeable enclosed container or packaging so that it ensures that the oxygen does not substantially penetrate within the container or packaging, therefore ensuring that the atmosphere within the enclosed container or packaging is essentially free of oxygen. By substantially gas exchange non-permeable enclosed container or packaging is understood that the container or packaging material does not allow, or allow a small number, of oxygen molecules to pass through it by permeation. The permeability of the container or packaging should be such that the oxygen content within the surrounding environment is less than 10%, preferably less than 5%, preferably less than 3%, preferably less than 0.1%, and preferably less than 0.01%, after at least one day of storage, preferably after at least 5 days of storage, preferably after at least 7 days of storage, preferably after at least 1 month of storage, preferably after 6 months of storage, and preferably after 12 months of storage. The amount of oxygen that permeates into a packaging material can be estimated (See Pharm. Dev. Technol. 2002, 7, 23-24, and Table 7). For example, Polyvivylchloride (PVC), Unoriented and Oriented polyethylene terephthalate (PET) materials show an oxygen transmission lower than 5 [cm³ mm/(m² day atm)], whereas polystyrene (PS) or polycarbonate (PC) show an oxygen transmission higher than 100 [cm³ mm/(m² day atm)].

The enclosed container or packaging for the retigabine, especially retigabine-free base, or pharmaceutical formulation thereof, in solid state as described herein is preferably a gas exchange non-permeable metal or glass or plastic container or packaging. For example, the enclosed container or packaging is a glass or plastic bottle, or a glass or plastic vial. More preferably, the enclosed gas exchange non-permeable container or packaging is an Aluminium foil bag or sachet.

In addition to permeation through container or packaging walls, the rate of oxygen ingress can be affected greatly by the container or packaging top, if present. Thus, it is also preferred that the top of the container or packaging may be also substantially gas exchange non-permeable. Also, it is preferred that the caps are sealed using an oxygen impermeable seal.

The retigabine, especially retigabine-free base, or pharmaceutical formulation thereof, in solid state as described herein can be firstly stored in a gas exchange permeable glass or plastic container or packaging (i.e. polyethylene plastic bag) which is turn stored within the gas exchange non-permeable metal or glass or plastic container or packaging, so as to avoid any potential damage (e.g. oxidation) of the gas exchange non-permeable metal or glass or plastic container or packaging to the retigabine, especially when the gas exchange non-permeable container or packaging is a metal container or packaging. Also, the gas exchange non-permeable metal or glass or plastic container or packaging can have an internal polymeric layer to avoid damage to the mixture, so that the intermediate gas exchange permeable glass or plastic container or packaging is not necessary.

The enclosed container or packaging for the pharmaceutical formulation comprising retigabine, especially retigabine-free base, in solid state as described herein is preferably selected from the group consisting of a non-permeable blister, such as an Al/Al blister, or an Al-polychloro-3-fluoroethylene homopolymer/PVC laminate blister, an aluminum foil, a glass or plastic bottle, and a glass or plastic vial.

It is also preferred that retigabine, especially retigabine-free base, in solid state and mixtures or formulations containing the same, are stored in the absence of light. Therefore, it is preferred that the enclosed container or packaging as hereinbefore described is also a substantially light non-permeable enclosed container or packaging.

According to the present invention there is further provided retigabine, especially retigabine-free base, obtained or obtainable by a process substantially as hereinbefore described.

There is also provided by the present invention a pharmaceutical formulation comprising an effective amount of retigabine substantially as hereinbefore described, together with a pharmaceutically acceptable carrier, diluent or excipient therefor. The term “effective amount” as used herein means an amount of retigabine which is capable of providing an anticonvulsive, antipyretic and/or analgesic therapeutic effect. By “pharmaceutically acceptable formulation” is meant that the carrier, diluent or excipient must be compatible with retigabine and not be deleterious to a recipient thereof. It can be generally understood in the art that the term pharmaceutically acceptable carrier comprises any carrier suitable for pharmaceutical use, including diluents and excipients. The one or more pharmaceutically acceptable carriers of the pharmaceutical formulation according to the present invention are at least one from the group consisting of pharmaceutically acceptable bulking agents, binders, disintegrants, lubricants, surfactants, drug delivery matrices, release modifying agents, glidants, diluents, vehicles, buffers, stabilizers, tonicity agents, coloring agents, flavouring agents, sweeteners, cryoprotectants, lyoprotectants, anti-oxidants, chelating agents, and preservatives. Pharmaceutically acceptable carriers are well known in the art and are described in, for example, Remington: The Science and Practice of Pharmacy, A. R. Gennaro, ed., Lippincott Williams & Wilkins; 20^{th} edition (Dec. 15, 2000).

Suitable pharmaceutically acceptable formulations according to the present invention are preferably in the form of solid compositions, such as a powder or lyophilized product for inclusion in a suspension or dispersion for an injectable formulation, or powder for an oral suspension, suppositories, tablets, coated tablets such as film coated tablets, non coated tablets, orodispersible tablets, pellets, pills, granules, capsules, or mini-tablets in capsules. Preferably, the solid pharmaceutical formulation of the invention is in the form of a film coated tablet.

The present invention further provides retigabine substantially as hereinbefore described, for use in the treatment of a disease state alleviated by administration of retigabine, in particular for the treatment of epilepsy. The present invention still further provides retigabine substantially as hereinbefore described, for use in the manufacture of a medicament for the treatment of a disease state alleviated by administration of retigabine, in particular for the treatment of epilepsy. The present invention also provides a method of treatment of a disease state alleviated by administration of retigabine, in particular epilepsy, which method comprises administering to the patient an effective amount of retigabine substantially as hereinbefore described.

According to the present invention, therefore, there is now provided a process of preparing retigabine, preferably retigabine free base, which process comprises reducing one or more —NO₂ groups as present in a compound of formula (II)

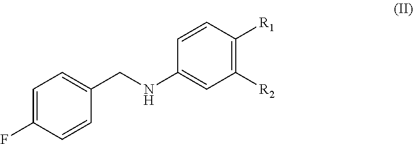

wherein:

R₁ is selected from the group consisting of —NH₂, —NO₂ and —NH—C(═O)—OCH₂CH₃; and

R₂ is either —NH₂ or —NO₂;

provided that at least one of R₁ and R₂ is —NO₂;

whereby in the case that R₁ represents —NH—C(═O)—OCH₂CH₃ in a compound of formula (II), retigabine is directly obtained further to the above reduction of a compound of formula (II); or

whereby in the case that R₁ represents —NH₂ or —NO₂ in a compound of formula (II), the above reduction of a compound of formula (II) provides an intermediate compound of formula (III)

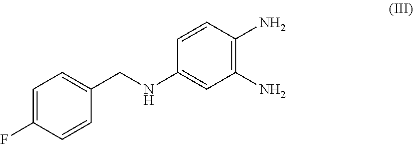 [SMILES: Nc1ccc(NCc2ccc(F)cc2)cc1N]

which compound of formula (III) is subsequently converted to retigabine;

characterized in that said reduction is carried out in the presence of a reducing agent and a weakly acidic proton donor.

More particularly, it is preferred that there is now provided by the present invention a process of preparing retigabine, preferably retigabine free base, which process comprises reducing one or more —NO₂ groups as present in a compound of formula (II)

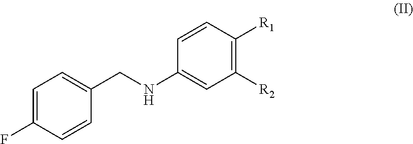

wherein:

R₁ is selected from the group consisting of —NH₂, —NO₂ and —NH—C(═O)—OCH₂CH₃; and

R₂ is either —NH₂ or —NO₂;

provided that at least one of R₁ and R₂ is —NO₂;

whereby in the case that R₁ represents —NH—C(═O)—OCH₂CH₃ in a compound of formula (II), retigabine is directly obtained further to the above reduction of a compound of formula (II); or

whereby in the case that R₁ represents —NH₂ or —NO₂ in a compound of formula (II), the above reduction of a compound of formula (II) provides an intermediate compound of formula (III)

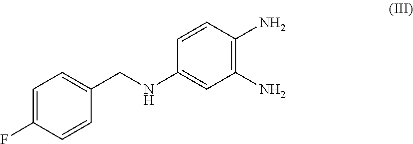 [SMILES: Nc1ccc(NCc2ccc(F)cc2)cc1N]

which compound of formula (III) is subsequently converted to retigabine;

characterized in that reduction is carried out in the presence of a reducing agent and a proton donor having a pKa value such that protonation of one or more of a compound of formula (II), and/or a compound of formula (III) if formed, and/or retigabine, is substantially avoided. It is particularly preferred that the proton donor has a pKa value that is at least greater than the pKa value of an amine group as present in retigabine; or in the case where a compound of formula (III) is formed it is particularly preferred that the proton donor has a pKa value that is greater than the pKa value of an amine group as present in a compound of formula (III). For the avoidance of doubt, reference herein to the “pKa of an amine group” denotes the pKa of the protonated form thereof.

It is preferred that in a compound of formula (II) above at least one, but not both, of R₁ and R₂ is —NO₂. A particularly preferred compound of formula (II) is one of the following compounds (IIa), (IIb) or (IIc):

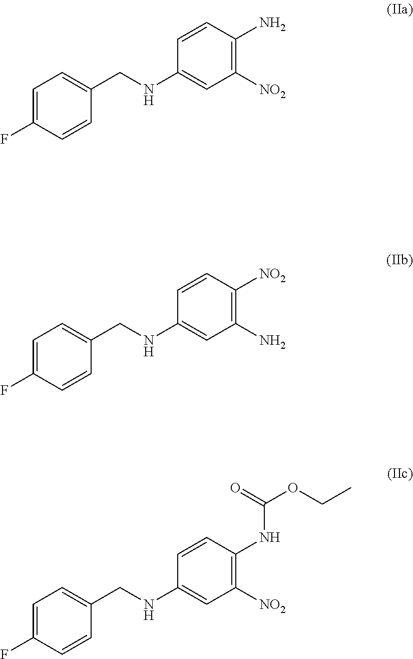 [SMILES: Nc1ccc(NCc2ccc(F)cc2)cc1[N+](=O)[O-] ; Nc1cc(NCc2ccc(F)cc2)ccc1[N+](=O)[O-] ; CCOC(=O)Nc1ccc(NCc2ccc(F)cc2)cc1[N+](=O)[O-]]

A process according to the present invention is advantageous since the reaction conditions employed avoid the use of a hydrogen atmosphere. The reduction reaction is carried out in the presence of a reduced metal and in the presence of a proton donor. Evolution of potential hydrogen gas (a potential by-product of the reaction) can be controlled by the addition rate of the proton donor and diluted with an inert gas, which thus facilitates carrying out the reduction process using conventional reactors.

A process according to the present invention is also advantageous since the reaction conditions employed avoid the use of strong acids that are commonly used as proton donors in combination with reducing agents, such as iron and zinc, in the prior art. For example, U.S. Pat. No. 5,384,330 describes that the reduction processes can be carried out with Zn/HCl or Fe/HCl. However, the use of strong acids as suggested in U.S. Pat. No. 5,384,330 is particularly detrimental for the synthesis of retigabine. More specifically, for a process as shown in Scheme 1 above of U.S. Pat. No. 5,384,330, if the reduction is carried out in the presence of a strong acid, this can be substantially consumed by protonation of the aniline amine groups in both the starting material and the intermediate product, thus requiring large amounts of base to neutralize before the subsequent acylation step. For a process as shown in Scheme 2 above of U.S. Pat. No. 5,384,330, retigabine dihydrochloride is directly obtained as a product if the reduction reaction is carried out in the presence of a strong acid, such as hydrochloric acid. Retigabine dihydrochloride is unstable even at temperatures of −18° C., which are below the reaction temperatures needed for the reduction processes carried out in aqueous media. The main degradant observed as a result of retigabine dihydrochloride instability is an internal cyclization by-product, namely 5-(4-fluorobenzylamino)-1,3-dihydrobenzimidazol-2-one, and it is expected that retigabine will also show similar instability in the presence of strong acids other than hydrochloric acid.

The pKa value for the aniline amine groups in retigabine is 5.25 (calculated using Advanced Chemistry Development ACD/Labs Software v8.19 for Solaris). The pKa value for the aniline amine groups in 4-(4-fluorobenzylamino)-1,2-phenylenediamine is 5.77 (calculated using Advanced Chemistry Development ACD/Labs Software v8.19 for Solaris). The pKa value for the less basic aniline amino group in 2-amino-5-(4-fluorobenzylamino)nitrobenzene (compound IIa) is 2.08 (calculated using Advanced Chemistry Development ACD/Labs Software v8.19 for Solaris). The pKa value for the less basic aniline amino group in 2-amino-4-(4-fluorobenzylamino)nitrobenzene (compound IIb) is 0.22 (calculated using Advanced Chemistry Development ACD/Labs Software v8.19 for Solaris).

Substantially as hereinbefore described, the inventors have now found that according to the present invention the reduction can be carried out in the presence of a weakly acidic proton donor preferably having a pKa value such that protonation of one or more of a compound of formula (II), and/or a compound of formula (III) if formed, and/or retigabine, is substantially avoided. It is further preferred that the pKa value of the proton donor is at least greater than the pKa value of an amine group as present in retigabine, or in the case where a compound of formula (III) is formed the pKa value of the proton donor is greater than the pKa value of an amine group as present in a compound of formula (III).

In a process according to the present invention which employs an intermediate compound of either formula (IIa) or (IIb) as above, it is desirable that the pKa of the weakly acidic proton donor is greater than the pKa value of an amine group as present in either a compound of formula (IIa), (IIb) or (III) and it is thus particularly preferred that the weakly acidic proton donor has a pKa value greater than about 5.7. In this way, a process according to the present invention that employs a compound of formula (IIa) or (IIb), and as such results in formation of an intermediate compound of formula (III), namely 4-(4-fluorobenzylamino)-1,2-phenylenediamine, in particular substantially avoids protonation of the latter. A process according to the present invention can thus avoid the requirement to employ large amounts of base to neutralize any protonated derivatives of a compound of formula (III) before conversion thereof to retigabine. Additionally, by employing a weakly acidic proton donor having a pKa value greater than about 5.7, this can also avoid formation of unstable protonated derivatives of retigabine.

In a process according to the present invention which employs an intermediate compound of formula (IIc) as above, it is desirable that the pKa of the weakly acidic proton donor is greater than the pKa value of an amine group as present in retigabine and it is thus particularly preferred that the weakly acidic proton donor has a pKa value greater than about 5.3. In this way, a process according to the present invention that employs a compound of formula (IIc) can avoid formation of unstable protonated derivatives of retigabine by using a weakly acidic proton donor having a pKa as above.

A particularly preferred weakly acidic proton donor for use in a process according to the present invention and having a pKa value as set out above is an ammonium salt, especially ammonium chloride. Ammonium chloride has a pKa value of about 9.3 and is a particularly preferred proton donor to be used in combination with zinc or iron in a process according to the present invention substantially as herein described.

In a process according to the present invention where an intermediate compound of formula (III) is formed, it is preferred that conversion thereof to retigabine involves reaction with an ethyl formate derivative, suitably represented by a compound of formula (IV)

CH₃CH₂OC(═O)X (IV)

wherein X represents a leaving group, such as a halo substituent, preferably chloro, or pyrocarbonic acid ethyl ester.

It is particularly preferred in a process according to the present invention to use finely powdered metals as the reducing agent so as to reduce the reaction time. Particularly preferred metals for a process according to the present invention are finely powdered zinc or iron, especially finely powdered zinc.

The European Medicines Agency in the “Guideline on the Specification Limits for Residues of Metal Catalysts or Metal Reagents,” reference EMEA/CHMP/SWP/4446/2000, aims to recommend maximum acceptable concentration limits for the residues of metal catalysts or metal reagents that may be present in pharmaceutical substances or in drug products. The metals addressed in the guideline are normally used as process catalysts or reagents during the synthesis of pharmaceutical substances. Their use may lead to residues in the final pharmaceutical substance, and consequently in the final drug product. Such metal residues do not provide any therapeutic benefit to the patient and should therefore be evaluated and restricted on the foundation of safety- and quality-based criteria. Metal residues are classified into three categories in the guideline based on their individual level of safety concern and concentration limits. The limits are based on the maximal daily dose, duration of treatment, and administration route of the drug product as well as the permitted daily exposure (PDE) of the metal residue.

The three categories are as follows:

Class 1 Metals: Metals of significant safety concern. This group includes metals that are known or suspect human carcinogens, or possible causative agents of other significant toxicity.

Class 2 metals: Metals of low safety concern. This group includes metals with lower toxic potential to man. They are generally well tolerated up to exposures that are typically encountered with administration of medicinal products. They may be trace metals required for nutritional purposes or they are often present in food stuffs or readily available nutritional supplements.

Class 3 metals: Metals of minimal safety concern. This group includes metals with no significant toxicity. Their safety profile is well established. They are generally well tolerated up to doses that are well beyond doses typically encountered with the administration of medicinal products. Typically they are ubiquitous in the environment or the plant and animal kingdoms.

Class 1 metals according to the above guideline include platinum, palladium, iridium, rhodium, ruthenium, osmium, molybdenum, nickel, chromium and vanadium. Class 2 metals according to the above guideline include copper and manganese. Class 3 metals according to the above guideline include iron and zinc.

As can be understood from the above, iron and zinc are classified as metals of minimal safety concern. Acceptable concentration limits in oral pharmaceutical compounds for metals that have been associated with prior art preparation of retigabine are summarized in Table 2 as follows:

TABLE 2

| | Concentration limit (ppm) |
|---|---|
| Metal residue | - Oral exposure - |
| Ni | 25 |
| Pd, Pt | 10 |
| Fe, Zn | 1300 |

A process according to the present invention that employs iron or zinc as a reducing agent provides retigabine with a content of residual metals much lower that the concentration limits accepted by the regulatory guidelines, without the need of further purification steps and/or use of metal scavengers. Furthermore, a process according to the present invention does not employ metals of significant or low safety concern and as such prepares retigabine that is free from carcinogenic risk. Accordingly, a process according to the present invention is further characterized by preparing retigabine, preferably retigabine free base, substantially free, or preferably completely free, from metals of significant or low safety concern, in particular retigabine, preferably retigabine free base, substantially free, or preferably completely free, from any of nickel, palladium or platinum. Furthermore, a process according to the present invention is further characterized by preparing retigabine, preferably retigabine free base, having a zinc or iron content within the regulatory requirements, and more particularly having a zinc or iron content of less than about 1300 ppm, more preferably less than about 500 ppm, and even more preferably less than 350 ppm.

There is also provided within the scope of the present invention a process of preparing retigabine free base, which process comprises reducing one or more —NO₂ groups as present in a compound of formula (II)

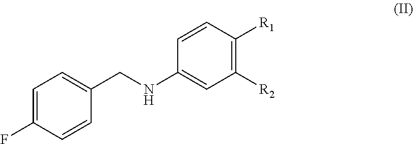

wherein:

R₁ is selected from the group consisting of —NH₂, —NO₂ and —NH—C(═O)—OCH₂CH₃; and

R₂ is either —NH₂ or —NO₂;

provided that at least one of R₁ and R₂ is —NO₂;

whereby in the case that R₁ represents —NH—C(═O)—OCH₂CH₃ in a compound of formula (II), retigabine free base is directly obtained further to the above reduction of a compound of formula (II); or

whereby in the case that R₁ represents —NH₂ or —NO₂ in a compound of formula (II), the above reduction of a compound of formula (II) provides an intermediate compound of formula (III)

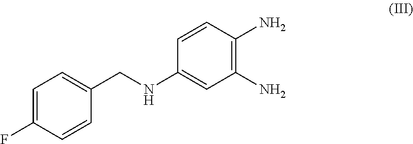 [SMILES: Nc1ccc(NCc2ccc(F)cc2)cc1N]

which compound of formula (III) is subsequently converted to retigabine-free base.

There is further provided within the scope of the present invention a process of preparing retigabine-free base, which process comprises reducing one or more —NO₂ groups as present in a compound of formula (II)

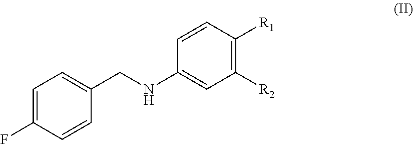

wherein:

R₁ is selected from the group consisting of —NH₂, —NO₂ and —NH—C(═O)—OCH₂CH₃; and

R₂ is either —NH₂ or —NO₂;

provided that at least one of R₁ and R₂ is —NO₂;

whereby in the case that R₁ represents —NH—C(═O)—OCH₂CH₃ in a compound of formula (II), retigabine-free base is directly obtained further to the above reduction of a compound of formula (II); or

whereby in the case that R₁ represents —NH₂ or —NO₂ in a compound of formula (II), the above reduction of a compound of formula (II) provides an intermediate compound of formula (III)

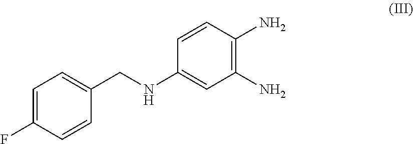 [SMILES: Nc1ccc(NCc2ccc(F)cc2)cc1N]

which compound of formula (III) is subsequently converted to retigabine-free base;

characterized in that neither protonated compound of formula (III) nor protonated retigabine are formed and/or isolated.

There is still further provided within the scope of the present invention a process of preparing retigabine-free base, which process comprises reacting an intermediate compound of formula (III)

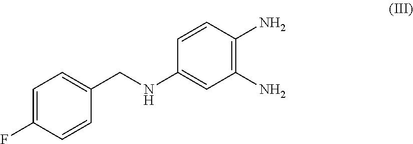 [SMILES: Nc1ccc(NCc2ccc(F)cc2)cc1N]

with an ethyl formate derivative, suitably represented by a compound of formula (IV)

CH₃CH₂C(═O)X (IV)

wherein X represents a leaving group, such as a halo substituent, preferably chloro, or pyrocarbonic acid ethyl ester, so as to provide retigabine-free base.

There is yet further provided within the scope of the present invention a process of preparing retigabine-free base, which process comprises reacting an intermediate compound of formula (III)

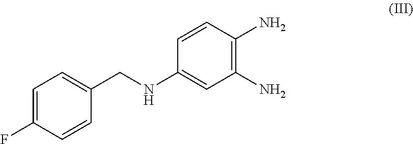 [SMILES: Nc1ccc(NCc2ccc(F)cc2)cc1N]

with an ethyl formate represented by a compound of formula (IV)

CH₃CH₂C(═O)X (IV)

wherein X represents a leaving group;

characterized in that neither protonated compound of formula (III) nor protonated retigabine are formed and/or isolated.

According to the present invention there is further provided retigabine, preferably retigabine-free base, obtained or obtainable by a process substantially as hereinbefore described. There is still further provided by the present invention retigabine, preferably retigabine free base, substantially free, or preferably completely free, from metals of significant or low safety concern, in particular retigabine, preferably retigabine-free base, substantially free, or preferably completely free, from any of nickel, palladium or platinum. There is still further provided by the present invention retigabine, preferably retigabine-free base, having a zinc or iron content within the regulatory requirements, and more particularly having a zinc or iron content of less than about 1300 ppm, more preferably less than about 500 ppm, and even more preferably less than 350 ppm. Advantageously, retigabine, preferably retigabine free base, according to the present invention has an overall purity of at least 99%.

The present invention further provides retigabine, preferably retigabine-free base, substantially as hereinbefore described, for use in the treatment of a disease state alleviated by administration of retigabine, in particular for the treatment of epilepsy. The present invention still further provides retigabine, preferably retigabine-free base, substantially as hereinbefore described, for use in the manufacture of a medicament for the treatment of a disease state alleviated by administration of retigabine, in particular for the treatment of epilepsy. The present invention also provides a method of treatment of a disease state alleviated by administration of retigabine, in particular epilepsy, which method comprises administering to the patient an effective amount of retigabine, preferably retigabine-free base, substantially as hereinbefore described.

The term “retigabine” as used herein is meant to comprise retigabine free base, or a pharmaceutically acceptable salt thereof.

The present invention will now be further illustrated by reference to the following Examples, which do not limit the scope of the invention in any way.

### EXAMPLES

### General Experimental Conditions

### HPLC Method:

The chromatographic separation was carried out in a Waters Sunfire C18, 5 μm, 4.6×250 mm column at 30° C.

The mobile phase was a filtered and degassed mixture of buffer solution and methanol (35:65). The buffer solution was prepared by dissolving about 0.5 mL of triethylamine in 500 mL of water, and then adjusting the pH to 7.1 with acetic acid.

The chromatograph was equipped with a 254 nm detector, and the flow rate was 0.8 mL per minute. The test samples (10 μL) were prepared by dissolving the appropriate amount of sample in mobile phase in order to obtain 1.0 mg per mL. The chromatogram was run for at least 45 minutes.

### Colorimetric Measurement:

Colorimetric measurements of the solid samples were obtained using a Minolta Chroma meter CR-300, using illuminant D65 and a measurement geometry of 2°.

The color quality (i.e., “whiteness”) of the retigabine samples was measured by depositing, leveling and measuring the sample without any special compacting treatment. Measurement was repeated three times for each sample. For characterization, the values of L*, a*, b* and Whiteness Index (WI) were specifically listed, each one being the mean of the three values available for each characterization parameter.

### Preparation of Crude Retigabine

### Crude Preparation 1:

A mixture of 170 g (0.65 mol) of 2-amino-4-(4-fluorobenzylamino)nitrobenzene and 340 g (5.3 mol) of zinc powder was suspended in 1.5 L of methanol under nitrogen atmosphere. A solution of 173.8 g (3.2 mol) of ammonium chloride in 600 mL of water was added dropwise under stirring, keeping the temperature below 35° C. The resulting mixture was stirred at room temperature for 4 hours. At this point, 166.4 g (1.29 mol) of diisopropylethylamine was added. Ethyl chloroformate (74.05 g, 0.68 mol) was added dropwise, keeping the temperature below 30° C. The mixture was stirred overnight at room temperature. Water (1.5 L) was added dropwise. The resulting mixture was stirred for 1 hour at room temperature, and the solvent was removed by filtration. The filtered solid was suspended in 1 L of ethyl acetate under nitrogen atmosphere. The suspension was stirred under heating and filtered. The filtered cake was suspended again in 1 L of ethyl acetate under nitrogen atmosphere. The suspension was stirred under heating and filtered. The filtered cake was suspended again in 1 L of ethyl acetate under nitrogen atmosphere. The suspension was stirred under heating and filtered. The solid was discarded and the combined filtrate was concentrated under reduced pressure to obtain 55 g ofretigabine. Purity (HPLC): 99.3%.

### Crude Preparation 2:

A mixture of 494 g (1.88 mol) of 2-amino-4-(4-fluorobenzylamino)nitrobenzene and 380 g (5.94 mol) of zinc powder was suspended in 3.5 L of methanol under nitrogen atmosphere. A solution of 530 g (9.91 mol) of ammonium chloride in 1.5 L of water was added dropwise under stirring, keeping the temperature below 30° C. The resulting mixture was stirred at 30° C. for 4 hours. At this point, 490 g (3.79 mol) of diisopropylethylamine was added. Ethyl chloroformate (221 g, 2.04 mol) was added dropwise, keeping the temperature below 10° C. The mixture was stirred overnight at room temperature. Water (4 L) was added dropwise. The resulting mixture was stirred for 1 hour at room temperature, and the solvent was removed by filtration. The filtered solid was suspended in 1.5 L of ethyl acetate under nitrogen atmosphere. The suspension was stirred at reflux temperature for 30 minutes, cooled down to 70° C. and filtered. The filtered cake was suspended again in 1.5 L of ethyl acetate under nitrogen atmosphere. The suspension was stirred at reflux temperature for 30 minutes, cooled down to 70° C. and filtered. The filtered cake was suspended again in 1.5 L of ethyl acetate under nitrogen atmosphere. The suspension was stirred at reflux temperature for 30 minutes, cooled down to 70° C. and filtered. The solid was discarded and the combined filtrate was concentrated under reduced pressure. The remaining residue was dissolved in ethyl acetate (700 ml) at reflux temperature, in the presence of active charcoal. The hot suspension was filtered and the resulting filtrate was cooled down to room temperature. Solvent was removed by filtration. The obtained solid was recrystallized with ethyl acetate (600 mL) to give 145 g of crude retigabine. Purity (HPLC): 99.8%.

### Crude Preparation 3:

A mixture of 502 g (1.91 mol) of 2-amino-4-(4-fluorobenzylamino)nitrobenzene and 653.9 g (10.2 mol) of zinc powder was suspended in 4.5 L of methanol under nitrogen atmosphere. A solution of 534.9 g (10.0 mol) of ammonium chloride in 1.5 L of water was added dropwise under stirring, keeping the temperature below 30° C. The resulting mixture was stirred at 30° C. for 4 hours. At this point, 470 g (3.64 mol) of diisopropylethylamine was added. Ethyl chloroformate (220 g, 2.03 mol) was added dropwise, keeping the temperature below 10° C. The mixture was stirred overnight at room temperature. Water (4.5 L) was added dropwise. The resulting mixture was stirred for 1 hour at room temperature, and the solvent was removed by filtration. The filtered solid was suspended in 2 L of ethyl acetate under nitrogen atmosphere. The suspension was stirred at reflux temperature for 30 minutes, cooled down to 70° C. and filtered. The filtered cake was suspended again in 2 L of ethyl acetate under nitrogen atmosphere. The suspension was stirred at reflux temperature for 30 minutes, cooled down to 70° C. and filtered. The filtered cake was suspended again in 2 L of ethyl acetate under nitrogen atmosphere. The suspension was stirred at reflux temperature for 30 minutes, cooled down to 70° C. and filtered. The solid was discarded and the combined filtrate was concentrated under reduced pressure. The remaining residue was dissolved in ethyl acetate (650 ml) at reflux temperature, in the presence of active charcoal. The hot suspension was filtered and the resulting filtrate was cooled down to room temperature. Solvent was removed by filtration. The obtained solid was recrystallized with ethyl acetate (600 mL) to give 182 g of crude retigabine. Purity (HPLC): 99.6%.

The results of the color quality measurements for the crude retigabine, using illuminant D65 (daylight) and a 2° angle of observation, are illustrated in Table 3, below:

TABLE 3

| | | | | | | Whiteness Index |
|---|---|---|---|---|---|---|
| L* | a* | b* | Y | x | y | (WI E313) |
| 75.44 | 2.06 | 8.90 | 48.98 | 0.3352 | 0.3472 | 0.04 |

### COMPARATIVE EXAMPLES

### Comparative Example 1

### Based on Example 1 of U.S. Pat. No. 6,538,151 B1

10 g of crude retigabine as obtained in crude preparation 1 and 0.68 g of active carbon were dissolved by warming with stirring in 30 mL of ethanol in a 100 mL reactor under nitrogen atmosphere. The solution was filtered hot through a pressure filter with stirring into a cooled 100 mL crystallizing reactor with 2 mL of ethanol such that the internal temperature in the crystallizing reactor was kept below 45° C. The remaining solution was then rinsed from the dissolving reactor through the pressure filter into the crystallizing reactor using 3 mL of hot ethanol and the suspension was swiftly cooled. It was subsequently stirred at 10° C. for 0.5 hours and the solid was filtered off with suction under nitrogen atmosphere. The product was washed three times with 5 mL of cooled ethanol each time. The crystallizate was then dried to weight constancy at 50-55° C. in a vacuum drying oven. 7.23 g (73% of theory) of retigabine was obtained. Purity (HPLC): 99.5%

The results of the color quality measurements for this sample of retigabine, using illuminant D65 (daylight) and a 2° angle of observation, are illustrated in Table 4, below:

TABLE 4

| | | | | | | Whiteness Index |
|---|---|---|---|---|---|---|
| L* | a* | b* | Y | x | y | (WI E313) |
| 86.48 | 0.90 | 5.94 | 68.95 | 0.3253 | 0.3402 | 39.83 |

### Comparative Example 2

### Based on Example 8 of U.S. Pat. No. 6,538,151 B1

10 g of crude retigabine as obtained in crude preparation 1 and 0.68 g of active carbon were dissolved by warming with stirring in 65 mL of isopropanol in a 250 mL reactor under nitrogen atmosphere. The solution was filtered hot through a pressure filter with stirring into a cooled 250 mL crystallizing reactor such that the internal temperature in the crystallizing reactor was kept at 60-65° C. The remaining solution was then rinsed from the dissolving reactor through the pressure filter into the crystallizing reactor using 8.5 mL of hot isopropanol and the suspension was swiftly cooled. It was subsequently stirred at 10° C. for 0.5 hours and the solid was filtered off with suction under nitrogen atmosphere. The product was washed three times with 8.5 mL of cooled isopropanol each time. The crystallizate was then dried to weight constancy at 50-55° C. in a vacuum drying oven. 7.15 g (72% of theory) of retigabine was obtained. Purity (HPLC): 99.5%.

The results of the color quality measurements for this sample of retigabine, using illuminant D65 (daylight) and a 2° angle of observation, are illustrated in Table 5, below:

TABLE 5

| | | | | | | Whiteness Index |
|---|---|---|---|---|---|---|
| L* | a* | b* | Y | x | y | (WI E313) |
| 89.65 | −0.10 | 3.70 | 75.55 | 0.3194 | 0.3362 | 57.95 |

### Comparative Example 3

Crude retigabine (5 g) as obtained in crude preparation 3 was suspended in isopropanol (15 mL). The suspension was heated to reflux temperature. The mixture was cooled down at 0-5° C. The solvent was removed by filtration. The filtered solid was washed with 2×5 mL of isopropanol and dried at 55° C. under reduced pressure. 4.71 g of retigabine was obtained. Yield: 94%. Purity (HPLC): 99.6%.

### Comparative Example 4

Crude retigabine (5 g) as obtained in crude preparation 3 was suspended in acetone (15 mL). The suspension was heated to reflux temperature. The mixture was cooled down to 0-5° C. The solvent was removed by filtration. The filtered solid was washed with 2×2 mL of acetone, and dried at 55° C. under reduced pressure. 1.87 g of retigabine was obtained. Yield: 37%. Purity (HPLC): 99.8%.

### Comparative Example 5

Crude retigabine (5 g) as obtained in crude preparation 3 was suspended in n-butanol (10 mL). The suspension was heated to reflux temperature. The mixture was cooled down to 0-5° C. and 5 mL of n-butanol was added. The solvent was removed by filtration. The filtered solid was washed with 2×2 mL of n-butanol, and dried at 55° C. under reduced pressure. 4.64 g of retigabine was obtained. Yield: 93%. Purity (HPLC): 99.6%.

### Comparative Example 6

Crude retigabine (5 g) as obtained in crude preparation 3 was suspended in toluene (10 mL). The suspension was heated to reflux temperature. The mixture was cooled down to 0-5° C. The solvent was removed by filtration. The filtered solid was washed with 2×4 mL of toluene, and dried at 55° C. under reduced pressure. 4.21 g of retigabine was obtained. Yield: 84%. Purity (HPLC): 99.6%.

### Comparative Example 7

Crude retigabine (5 g) as obtained in crude preparation 3 was suspended in ethanol (10 mL). The suspension was heated to reflux temperature. The mixture was cooled down to 0-5° C. and 5 mL of ethanol was added. The solvent was removed by filtration. The filtered solid was washed with 2×2 mL of ethanol, and dried at 55° C. under reduced pressure. 4.73 g of retigabine was obtained. Yield: 95%. Purity (HPLC): 99.6%.

### Retigabine Preparation According to the Present Invention

### Example 1

Crude retigabine (5 g) as obtained in crude preparation 2 was suspended in a mixture of isopropanol (10 mL) and ethyl acetate (2 mL). The suspension was heated to reflux temperature. The resulting mixture was cooled down to 20-25° C., and a mixture of isopropanol (2 mL) and ethyl acetate (0.4 mL) was added. The solvent was removed by filtration. The solid was washed with 2×2 ml of isopropanol and dried under reduced pressure at 60° C. 4.69 g of retigabine was obtained. Purity (HPLC): 99.9%.

The results of the color quality measurements for this sample of retigabine, using illuminant D65 (daylight) and a 2° angle of observation, are illustrated in Table 6, below:

TABLE 6

| | | | | | | Whiteness Index |
|---|---|---|---|---|---|---|
| L* | a* | b* | Y | x | y | (WI E313) |
| 94.40 | 0.02 | 1.77 | 86.20 | 0.3158 | 0.3323 | 78.11 |

### Example 2

Crude retigabine (5 g) as obtained in crude preparation 3 was suspended in a mixture of isopropanol (8 mL) and ethyl acetate (2 mL). The suspension was heated to reflux temperature. The mixture was cooled down to 0-5° C. and 2 mL of isopropanol was added. The solvent was removed by filtration. The filtered solid was washed with 2×2 mL of a mixture (1:1) of isopropanol and ethyl acetate, and with 2×3 mL of isopropanol. The solid was dried at 60° C. under reduced pressure. 4.32 g of retigabine was obtained. Yield: 86%. Purity (HPLC): 99.7%.

### Color and HPLC Purity Measurements

Test 1: Evaluation of the Variation of the Color Quality and the HPLC Chemical Purity of Retigabine after Crystallization or Slurrying Processes.

Color Measurement Test 1:

The results of the color quality measurements for samples of retigabine obtained in above Examples 1 and 2 according to the present invention and Comparative Examples 3 to 7 using illuminant D65 (daylight) and a 2° angle of observation, are illustrated in Table 7, below:

TABLE 7

| | | | | | | | | Whiteness |
|---|---|---|---|---|---|---|---|---|
| | | | | | | | | Index |
| Example | Solvent | L* | a* | b* | Y | x | y | (WI E313) |
| 1 (Invention) | Isopropanol- | 94.49 | 0.02 | 1.77 | 86.20 | 0.3158 | 0.3323 | 78.11 |
| | Ethyl | | | | | | | |
| | acetate | | | | | | | |
| 2 (Invention) | Isopropanol- | 90.28 | 0.93 | 3.41 | 76.91 | 0.3203 | 0.3349 | 60.80 |
| | Ethyl | | | | | | | |
| | acetate | | | | | | | |
| Comparative | Isopropanol | 88.98 | 0.57 | 3.79 | 74.12 | 0.3205 | 0.3360 | 55.98 |
| Example 3 | | | | | | | | |
| Comparative | Acetone | 92.75 | 0.66 | 7.54 | 82.40 | 0.3271 | 0.3427 | 47.59 |
| Example 4 | | | | | | | | |
| Comparative | n-Butanol | 87.57 | 1.36 | 5.04 | 71.17 | 0.3242 | 0.3380 | 46.67 |
| Example 5 | | | | | | | | |
| Comparative | Toluene | 85.73 | 2.08 | 3.81 | 67.45 | 0.3231 | 0.3351 | 48.76 |
| Example 6 | | | | | | | | |
| Comparative | Ethanol | 89.63 | 0.90 | 3.98 | 75.51 | 0.3213 | 0.3361 | 55.56 |
| Example 7 | | | | | | | | |

HPLC Purity Measurement Test 1:

The results of the HPLC chemical purity measurements for samples of retigabine obtained in above Examples 1 and 2 according to the present invention and Comparative Examples 3 to 7 (HPLC Method), are illustrated in Table 8, below:

TABLE 8

| | | | Reduction |
|---|---|---|---|
| | Initial purity | Final purity | of total |
| Example | (HPLC %) | (HPLC %) | impurities (%) |
| 1 (Invention) | 99.8 | 99.9 | 50% |
| 2 (Invention) | 99.6 | 99.7 | 25% |
| Comparative Example 1 | 99.3 | 99.5 | 29% |
| Comparative Example 2 | 99.3 | 99.5 | 29% |
| Comparative Example 3 | 99.6 | 99.6 | 0% |
| Comparative Example 4 | 99.6 | 99.8 | 50% |
| Comparative Example 5 | 99.6 | 99.6 | 0% |
| Comparative Example 6 | 99.6 | 99.6 | 0% |
| Comparative Example 7 | 99.6 | 99.6 | 0% |

Test 2: Evaluation of the Variation of the Color Quality and the HPLC Chemical Purity of Retigabine after Different Drying Processes.

Retigabine as obtained in comparative Example 3 was wet with some drops of isopropanol. The wet solid was stressed at 55° C. and 40° C. for three days, and at 25° C. for four days, in closed vials under inert atmosphere (i.e. nitrogen). Then, vials were opened and the solvent was removed under reduced pressure. Dried samples were grinded and homogenized before analysis.

Colour Measurement Test 2:

The results of the color quality measurements for these samples of retigabine, using illuminant D65 (daylight) and a 2° angle of observation, are illustrated in Table 9, below:

TABLE 9

| | | | | | | | Whiteness | |
|---|---|---|---|---|---|---|---|---|
| Stress | | | | | | | Index | WI |
| temp. | L* | a* | b* | Y | x | y | (WI E313) | worsening |
| Initial | 88.98 | 0.57 | 3.79 | 74.12 | 0.3205 | 0.3360 | 55.98 | — |
| 55° C. | 79.51 | 1.89 | 5.62 | 55.82 | 0.3272 | 0.3395 | 26.37 | −53% |
| 40° C. | 84.31 | 1.66 | 5.71 | 64.66 | 0.3263 | 0.3394 | 36.10 | −36% |
| 25° C. | 89.15 | 0.88 | 4.29 | 74.48 | 0.3219 | 0.3367 | 54.03 | −3% |

HPLC Purity Measurement Test 2:

The results of the HPLC purity measurements for these samples of retigabine (HPLC Method), are illustrated in Table 10, below:

TABLE 10

| | | Purity (HPLC %) |
|---|---|---|
| Stress temp. | Purity (HPLC %) | worsening |
| Initial | 99.6 | — |
| 55° C. | 99.7 | +0.1% |
| 40° C. | 99.6 | 0% |
| 25° C. | 99.6 | 0% |

Test 3: Evaluation of the Variation of the Color Quality and the HPLC Chemical Purity Ofretigabine after Different Drying Processes.

Retigabine as obtained in comparative Example 5 was wet with some drops of n-butanol. The wet solid was stressed at 55° C. and 40° C. for three days, and at 25° C. for four days, in closed vials under inert atmosphere (i.e. nitrogen). Then, vials were opened and the solvent was removed under reduced pressure. Dried samples were grinded and homogenized before analysis.

Color Measurement Test 3:

The results of the color quality measurements for these samples of retigabine, using illuminant D65 (daylight) and a 2° angle of observation, are illustrated in Table 11, below:

TABLE 11

| | | | | | | | Whiteness | |
|---|---|---|---|---|---|---|---|---|
| Stress | | | | | | | Index | WI |
| temp. | L* | a* | b* | Y | x | y | (WI E313) | worsening |
| Initial | 87.57 | 1.36 | 5.04 | 71.17 | 0.3242 | 0.3380 | 46.67 | — |
| 55° C. | 56.22 | 6.77 | 4.49 | 24.13 | 0.3391 | 0.3345 | −6.39 | −114% |
| 40° C. | 85.72 | 1.60 | 5.50 | 67.43 | 0.3256 | 0.3389 | 40.28 | −14% |
| 25° C. | 87.42 | 1.53 | 5.14 | 70.87 | 0.3246 | 0.3381 | 45.88 | −2% |

HPLC Purity Measurement Test 3:

The results of the HPLC purity measurements for these samples of retigabine (HPLC Method), are illustrated in Table 12, below:

TABLE 12

| | | Purity (HPLC %) |
|---|---|---|
| Stress temp. | Purity (HPLC %) | worsening |
| Initial | 99.6 | — |
| 55° C. | 99.3 | −0.3% |
| 40° C. | 99.6 | 0% |
| 25° C. | 99.6 | 0% |

Test 4: Evaluation of the Variation of the Color Quality and the HPLC Chemical Purity of Retigabine after Different Drying Processes.

Retigabine as obtained in comparative Example 6 was wet with some drops of toluene. The wet solid was stressed at 55° C. and 40° C. for three days, and at 25° C. for four days, in closed vials under inert atmosphere (i.e. nitrogen). Then, vials were opened and the solvent was removed under reduced pressure. Dried samples were grinded and homogenized before analysis.

Color Measurement Test 4:

The results of the color quality measurements for these samples of retigabine, using illuminant D65 (daylight) and a 2° angle of observation, are illustrated in Table 13, below:

TABLE 13

| | | | | | | | Whiteness | |
|---|---|---|---|---|---|---|---|---|
| Stress | | | | | | | Index | WI |
| temp. | L* | a* | b* | Y | x | y | (WI E313) | worsening |
| Initial | 85.73 | 2.08 | 3.81 | 67.45 | 0.3231 | 0.3351 | 48.76 | — |
| 55° C. | 79.91 | 2.32 | 5.12 | 56.52 | 0.3268 | 0.3381 | 29.77 | −39% |
| 40° C. | 83.46 | 2.21 | 4.64 | 63.03 | 0.3252 | 0.3369 | 39.60 | −19% |
| 25° C. | 85.72 | 1.85 | 3.84 | 67.43 | 0.3228 | 0.3354 | 48.47 | −1% |

HPLC Purity Measurement Test 4:

The results of the HPLC purity measurements for these samples of retigabine (HPLC Method), are illustrated in Table 14, below:

TABLE 14

| | | Purity (HPLC %) |
|---|---|---|
| Stress temp. | Purity (HPLC %) | worsening |
| Initial | 99.6 | — |
| 55° C. | 99.6 | 0% |
| 40° C. | 99.4 | −0.2% |
| 25° C. | 99.6 | 0% |

Test 5: Evaluation of the Variation of the Color Quality and the HPLC Chemical Purity of Retigabine after Different Storage Conditions.

Retigabine as obtained in Example 1 according to the present invention was exposed to light in a closed, transparent glass vial under inert atmosphere (i.e. nitrogen); and to air in an opened, brown glass vial covered with aluminum foil, at room temperature. Samples were grinded, homogenized and analyzed after seven days.

Color Measurement Test 5:

The results of the color quality measurements for these samples of retigabine, using illuminant D65 (daylight) and a 2° angle of observation, are illustrated in Table 15, below:

TABLE 15

| | | | | | | | Whiteness | |
|---|---|---|---|---|---|---|---|---|
| | | | | | | | Index | WI |
| Stress cond. | L* | a* | b* | Y | x | y | (WI E313) | worsening |
| Initial | 94.40 | 0.02 | 1.77 | 86.20 | 0.3158 | 0.3323 | 78.11 | — |
| Light (inert | 93.97 | −0.29 | 1.73 | 85.20 | 0.3153 | 0.3324 | 77.34 | −1% |
| atmosphere) | | | | | | | | |
| Air (absence | 85.00 | −2.01 | 6.16 | 66.01 | 0.3216 | 0.3430 | 35.09 | −55% |
| of light) | | | | | | | | |

HPLC Purity Measurement Test 5:

The results of the HPLC purity measurements for these samples of retigabine (HPLC Method), are illustrated in Table 16, below:

TABLE 16

| | | Purity (HPLC %) |
|---|---|---|
| Stress cond. | Purity (HPLC %) | worsening |
| Initial | 99.9% | — |
| Light (inert atmosphere) | 99.9% | 0% |
| Air (absence of light) | 99.0% | −1% |

### Intermediate Preparation

### Preparation of 2-amino-4-(4-fluorobenzylamino)nitrobenzene

Synthesis A:

A mixture of 5-chloro-2-nitroaniline (431.5 g, 2.5 mol), 4-fluorobenzylamine (938.6 g, 7.5 mol), triethylamine (303.6 g, 3.0 mol) and iodine (12.7 g, 79 mmol) was suspended in 2 L of dimethylsulfoxide. The mixture was heated to 115-120° C. for 72 hours, then cooled to room temperature and poured into 6 L of water at 0° C. and stirred for 3 hours. The resulting mixture was filtered off, and the filtered solid was recrystallized from toluene and dried under reduced pressure to obtain 494 g of 2-amino-4-(4-fluorobenzylamino)nitrobenzene. Yield: 76%.

Synthesis B (Scale Up of Synthesis A):

A mixture of 5-chloro-2-nitroaniline (863.0 g, 5.0 mol), 4-fluorobenzylamine (1.87 Kg, 15.0 mol), triethylamine (657.8 g, 6.5 mol) and iodine (25.4 g, 158 mmol) was suspended in 4 L of dimethylsulfoxide. The mixture was heated to 115-120° C. for 72 hours, then cooled to room temperature and poured into 12 L of water at 0° C. and stirred for 3 hours. The resulting mixture was filtered off, and the filtered solid was recrystallized from toluene and dried under reduced pressure to obtain 980 g of 2-amino-4-(4-fluorobenzylamino)nitrobenzene. Yield: 75%.

### Retigabine Preparation

### Example 3

A suspension of 5.2 g (20 mmol) of 2-amino-4-(4-fluorobenzylamino)nitrobenzene in 100 mL of 1,4-dioxane was hydrogenated at 55-60° C. under normal pressure in the presence of 2 g of Raney nickel. After 27 hours the catalyst was filtered off under nitrogen atmosphere. To the resulting solution was added 3.2 g (25 mmol) of diisopropylethylamine. A solution of 2.3 g (21 mmol) of ethyl chloroformate in 15 mL of 1,4-dioxane was added dropwise and stirred at 10-20° C. under nitrogen atmosphere. The mixture was stirred for 2 hours at room temperature. Water (200 mL) was added dropwise, and a solid material gradually crystallized out the solution. The resulting mixture was stirred for 1 hour at room temperature, and the solvent was removed by filtration. The filtered solid was suspended in 100 mL of ethyl acetate under heating, and the resulting mixture was filtered. The filtered solution was concentrated under reduced pressure, and the remaining residue was recrystallized from ethyl acetate to obtain 1.3 g of retigabine in the form of free base. Yield: 21%. Purity (HPLC): 92.7%. Residual Ni (ICP-MS): 497±89 ppm (regulatory limit: 25 ppm).

### Example 4

A suspension of 5.2 g (20 mmol) of 2-amino-4-(4-fluorobenzylamino)nitrobenzene in 100 mL of 1,4-dioxane was hydrogenated at 55-60° C. under normal pressure in the presence of 2 g of 5% palladium on charcoal. After 120 hours the catalyst was filtered off under nitrogen atmosphere. To the resulting solution was added 3.2 g (25 mmol) of diisopropylethylamine. A solution of 2.3 g (21 mmol) of ethyl chloroformate in 15 mL of 1,4-dioxane was added dropwise and stirred at 10-20° C. under nitrogen atmosphere. The mixture was stirred for 2 hours at room temperature. Water (200 mL) was added dropwise, and a solid material gradually crystallized out the solution. The resulting mixture was stirred for 1 hour at room temperature, and the solvent was removed by filtration. The filtered solid was suspended in 100 mL of ethyl acetate under heating, and the resulting mixture was filtered. The filtered solution was concentrated under reduced pressure, and the remaining residue was recrystallized from ethyl acetate to obtain 1.2 g of retigabine in the form of free base. Yield: 20%. Purity (HPLC): 99.3%. Residual Pd (ICP-MS): 11±2 ppm (regulatory limit: 10 ppm).

### Example 5

A mixture of 5.2 g (20 mmol) of 2-amino-4-(4-fluorobenzylamino)nitrobenzene and 6.5 g (100 mmol) of zinc powder was suspended in 45 mL of methanol under nitrogen atmosphere. A solution of 5.3 g (100 mmol) of ammonium chloride in 15 mL of water was added dropwise under stirring, keeping the temperature below 30° C. The resulting mixture was stirred at room temperature for 4 hours. At this point, 3.2 g (25 mmol) of diisopropylethylamine was added. Ethyl chloroformate (2.3 g, 20 mmol) was added dropwise and stirred at 10-20° C. under nitrogen atmosphere. The mixture was stirred for 3 hours at room temperature. Water (200 mL) was added dropwise. The resulting mixture was stirred for 1 hour at room temperature, and the solvent was removed by filtration. The filtered solid was suspended in 50 mL of ethyl acetate under heating, and the resulting mixture was filtered. The filtered solution was concentrated under reduced pressure, and the remaining residue was recrystallized from ethyl acetate to obtain 0.9 g of retigabine in the form of free base. Yield: 15%. Purity (HPLC): 99.6%. Residual Zn (ICP-MS): 238±73 ppm (regulatory limit: 1300 ppm).

### Example 6

A mixture of 5.0 g (15 mmol) of 2-ethoxycarbonylamino-5-(4-fluorobenzylamino)-nitrobenzene and 4.9 g (75 mmol) of zinc powder was suspended in 30 mL of methanol under nitrogen atmosphere. A solution of 4.0 g (75 mmol) of ammonium chloride in 15 mL of water was added dropwise under stirring, keeping the temperature below 30° C. The resulting mixture was stirred at room temperature for 4 hours. Water (150 mL) was added dropwise. The resulting mixture was stirred for 1 hour at room temperature, and the solvent was removed by filtration. The filtered solid was suspended in 100 mL of ethyl acetate under heating, and the resulting mixture was filtered. The filtered solution was concentrated under reduced pressure, and the remaining residue was recrystallized from ethyl acetate to obtain 0.6 g of retigabine in the form of free base. Yield: 13%. Purity (HPLC): 99.1%. Residual Zn (ICP-MS): 85±8 ppm Zn (regulatory limit: 1300 ppm).

### Example 7

A mixture of 494 g (1.88 mol) of 2-amino-4-(4-fluorobenzylamino)nitrobenzene and 380 g (5.94 mol) of zinc powder was suspended in 3.5 L of methanol under nitrogen atmosphere. A solution of 530 g (9.91 mol) of ammonium chloride in 1.5 L of water was added dropwise under stirring, keeping the temperature below 30° C. The resulting mixture was stirred at 30° C. for 4 hours. At this point, 490 g (3.79 mol) of diisopropylethylamine was added. Ethyl chloroformate (221 g, 2.04 mol) was added dropwise, keeping the temperature below 10° C. The mixture was stirred overnight at room temperature. Water (4 L) was added dropwise. The resulting mixture was stirred for 1 hour at room temperature, and the solvent was removed by filtration. The filtered solid was suspended in 1.5 L of ethyl acetate under nitrogen atmosphere. The suspension was stirred at reflux temperature for 30 minutes, cooled down to 70° C. and filtered. The filtered cake was suspended again in 1.5 L of ethyl acetate under nitrogen atmosphere. The suspension was stirred at reflux temperature for 30 minutes, cooled down to 70° C. and filtered. The filtered cake was suspended again in 1.5 L of ethyl acetate under nitrogen atmosphere. The suspension was stirred at reflux temperature for 30 minutes, cooled down to 70° C. and filtered. The solid was discarded and the combined filtrate was concentrated under reduced pressure. The remaining residue was dissolved in ethyl acetate (700 ml) at reflux temperature, in the presence of active charcoal. The hot suspension was filtered and the resulting filtrate was cooled down to room temperature. Solvent was removed by filtration. The obtained solid was recrystallized with ethyl acetate (600 mL) to give 145 g of retigabine. Yield: 25%. Purity (HPLC): 99.8%. Residual Zn (ICP-MS): 11.9 ppm Zn (regulatory limit: 1300 ppm).

### Example 8

A mixture of 502 g (1.91 mol) of 2-amino-4-(4-fluorobenzylamino)nitrobenzene and 653.9 g (10.2 mol) of zinc powder was suspended in 4.5 L of methanol under nitrogen atmosphere. A solution of 534.9 g (10.0 mol) of ammonium chloride in 1.5 L of water was added dropwise under stirring, keeping the temperature below 30° C. The resulting mixture was stirred at 30° C. for 4 hours. At this point, 470 g (3.64 mol) of diisopropylethylamine was added. Ethyl chloroformate (220 g, 2.03 mol) was added dropwise, keeping the temperature below 10° C. The mixture was stirred overnight at room temperature. Water (4.5 L) was added dropwise. The resulting mixture was stirred for 1 hour at room temperature, and the solvent was removed by filtration. The filtered solid was suspended in 2 L of ethyl acetate under nitrogen atmosphere. The suspension was stirred at reflux temperature for 30 minutes, cooled down to 70° C. and filtered. The filtered cake was suspended again in 2 L of ethyl acetate under nitrogen atmosphere. The suspension was stirred at reflux temperature for 30 minutes, cooled down to 70° C. and filtered. The filtered cake was suspended again in 2 L of ethyl acetate under nitrogen atmosphere. The suspension was stirred at reflux temperature for 30 minutes, cooled down to 70° C. and filtered. The solid was discarded and the combined filtrate was concentrated under reduced pressure. The remaining residue was dissolved in ethyl acetate (650 ml) at reflux temperature, in the presence of active charcoal. The hot suspension was filtered and the resulting filtrate was cooled down to room temperature. Solvent was removed by filtration. The obtained solid was recrystallized with ethyl acetate (600 mL) to give 182 g of retigabine. Yield: 31%. Purity (HPLC): 99.5%.

### Example 8

A mixture of 52 g (0.2 mol) of 2-amino-4-(4-fluorobenzylamino)nitrobenzene, 56 g (1 mol) of iron powder and 53 g (1 mol) of ammonium chloride was suspended in a mixture of 450 mL of methanol and 150 mL of water. The resulting mixture was stirred at reflux temperature for 4 hours. At this point, the mixture was cooled to 10-20° C. and 32 g (0.25 mol) of diisopropylethylamine was added. Ethyl chloroformate (23 g, 0.21 mol) was added dropwise and stirred at 10-20° C. under nitrogen atmosphere. The mixture was stirred for 16 hours at room temperature. Water (2 L) was added dropwise. The resulting mixture was stirred for 1 hour at room temperature, and the solvent was removed by filtration. The filtered solid was suspended in 500 mL of ethyl acetate under heating, and the resulting mixture was filtered. The filtered solution was concentrated under reduced pressure, and the remaining residue was recrystallized from ethyl acetate (100 mL) to obtain 10.5 g of retigabine in the form of free base. Yield: 17.3%. Purity (HPLC): 99%.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-58. (canceled)

59. Solid state retigabine free base in an inert atmosphere.

60. The retigabine of claim 59, wherein the retigabine has a Whiteness Index of at least about 60.

61. The retigabine of claim 59, wherein the retigabine comprises up to 99% (w/w) of amorphous form retigabine.

62. The retigabine of claim 59, wherein the retigabine is in crystalline form.

63. The retigabine of claim 59, wherein the inert atmosphere is selected from the group consisting of a vacuum and an inert gas atmosphere.

64. The retigabine of claim 63, wherein the inert gas is selected from the group consisting of nitrogen, argon, and carbon dioxide.

65. The retigabine of claim 59, wherein the inert atmosphere is essentially free of oxygen.

66. The retigabine of claim 65, wherein the inert atmosphere includes an oxygen scavenger.

67. Solid state retigabine in a package comprising an inert atmosphere.

68. The retigabine of claim 67, wherein the inert atmosphere is selected from the group consisting of a vacuum and an inert gas atmosphere.

69. The retigabine of claim 68, wherein the inert gas is selected from the group consisting of nitrogen, argon, and carbon dioxide.

70. The retigabine of claim 67, wherein the inert atmosphere is essentially free of oxygen.

71. The retigabine of claim 70, wherein the inert atmosphere includes an oxygen scavenger.

72. The retigabine of claim 67, wherein the package is substantially non-permeable to oxygen.

73. The retigabine of claim 67, wherein the package is selected from the group consisting of a glass bottle, a glass vial, a plastic bottle, a plastic vial, a foil bag, and a foil sachet.

74. The retigabine of claim 67, wherein the package is substantially non-permeable to light.

75. The retigabine according to claim 67, wherein the retigabine is retigabine free base.

76. A pharmaceutical formulation comprising solid state retigabine and one or more pharmaceutically acceptable carriers, wherein the formulation is in an inert atmosphere.

77. The pharmaceutical formulation of claim 76, wherein the retigabine comprises up to 99% (w/w) of amorphous form retigabine.

78. The pharmaceutical formulation of claim 76, wherein the retigabine is in crystalline form.

79. The pharmaceutical formulation of claim 76, wherein the inert atmosphere is selected from the group consisting of a vacuum and an inert gas atmosphere.

80. The pharmaceutical formulation of claim 79, wherein the inert gas is selected from the group consisting of nitrogen, argon, and carbon dioxide.

81. The pharmaceutical formulation of claim 76, wherein the inert atmosphere is essentially free of oxygen.

82. The pharmaceutical formulation of claim 81, wherein the inert atmosphere includes an oxygen scavenger.

83. The pharmaceutical formulation of claim 76, wherein the retigabine is retigabine free base.

84. A pharmaceutical formulation comprising solid state retigabine and one or more pharmaceutically acceptable carriers, in a package comprising an inert atmosphere.

85. The pharmaceutical formulation of claim 84, wherein the inert atmosphere is selected from the group consisting of a vacuum and an inert gas atmosphere.

86. The pharmaceutical formulation of claim 85, wherein the inert gas is selected from the group consisting of nitrogen, argon, and carbon dioxide.

87. The pharmaceutical formulation of claim 84, wherein the inert atmosphere is essentially free of oxygen.

88. The pharmaceutical formulation of claim 87, wherein the inert atmosphere includes an oxygen scavenger.

89. The pharmaceutical formulation of claim 84, wherein the package is substantially non-permeable to oxygen.

90. The pharmaceutical formulation of claim 84, wherein the package is substantially non-permeable to light.

91. The pharmaceutical formulation of claim 84, wherein the package is selected from the group consisting of a glass bottle, a glass vial, a plastic bottle, a plastic vial, a foil bag, and a foil sachet.

92. The pharmaceutical formulation according to claim 84, wherein the retigabine is retigabine free base.
